(19) United States
(12) Patent Application Publication
(10) Pub. No.: US 2006/0062866 A1
(43) Pub. Date: Mar. 23, 2006

(54) Starch-pomegranate juice complex as an HIV entry inhibitor and topical microbicide

(75) Inventors: Alexander Robert Neurath, New York, NY (US); Nathan Strick, Oceanside, NY (US); Yun-Yao Li, Fresh Meadows, NY (US)

Correspondence Address: FRISHAUF, HOLTZ, GOODMAN & CHICK, PC, 220 Fifth Avenue, 16TH Floor, NEW YORK, NY, 10001-7708, US

(73) Assignee: NEW YORK BLOOD CENTER, INC., New York, NY (US)

(21) Appl. No.: 11/222,989

(22) Filed: Sep. 9, 2005

Related U.S. Application Data
(60) Provisional application No. 60/611,778, filed on Sep. 21, 2004.

(51) Int. Cl. A61K 36/185

(52) U.S. Cl. 424/769; 514/60

(57) ABSTRACT

A complex comprising a starch and an active anti-HIV-1 or anti-HIV-2 ingredient of pomegranate juice that is adsorbed on the starch when the starch is in a water insoluble form. The complex inhibits HIV-1 or HIV-2 infection and blocks the binding of HIV-1 or HIV-2 to the CD4 receptor and the CCR5 and CXCR4 coreceptors. The complex is used in a method of preventing HIV-1 or HIV-2 infection comprising administering to a mucous membrane of a human a pharmaceutically effective anti-HIV-1 or anti-HIV-2 amount of the complex.

### CROSS-REFERENCE TO RELATED APPLICATION

This application claims the benefit of priority under 35 USC 119(e) for U.S. provisional application Ser. No. 60/611,778 filed Sep. 21, 2004, the entire contents of which are incorporated by reference herein.

### GOVERNMENT RIGHTS

This invention was made with United States government support under Grant P01 HD41761 from the National Institute of Health (“NIH”). The United States government may have certain rights in this invention.

### BACKGROUND OF THE INVENTION

1. Field of the Invention

The present invention concerns a starch-pomegranate juice complex that can be used as an HIV-1 or HIV-2 entry inhibitor and as a topical microbicide and a method for preventing HIV-1 or HIV-2 infection by administering the complex vaginally to a woman.

2. Background of the Invention

For approximately 24 years, the acquired immunodeficiency syndrome (AIDS) pandemic has claimed approximately 30 million lives, causing about 14,000 new human immunodeficiency virus type (HIV-1) infections daily worldwide in 2003. About 80% of infections occur by heterosexual transmission. In the absence of vaccines, topical microbicides expected to block virus transmission offer hope for controlling the pandemic. Antiretroviral chemotherapeutics have decreased AIDS mortality in industrialized countries, but only minimally in developing countries. To prevent an analogous dichotomy, microbicides should be acceptable, accessible, affordable and accelerative in transition from development to marketing. Already marketed pharmaceutical excipients or foods, with established safety records and adequate anti-HIV-1 activity may provide this option.

The global AIDS epidemic has proceeded relentlessly for approximately 24 years with no promising prophylactic intervention in sight. In 2003, there were 5 million new HIV infections and 3 million AIDS deaths [UNAIDS: AIDS Epidemic Update (December 2003) 2004 [http://www.unaids.org/en/other/functionalities/View Document.asp?href=http%3a%2f%2fgva-doc-owl%2fWEBcontent%2fDocuments%2fpub%2fPublications%2fIRC-pub06%2fJC943-EpiUpdate2003en%26%2346%3bpdf]]. To date the number of individuals living with HIV-1 infection/AIDS has reached 40 million, and as discussed above, approximately 30 million people have already died from AIDS since the beginning of the pandemic [UNAIDS: AIDS Epidemic Update (December 2003), 2004; WHO/SEARO CDS HIV/AIDS: End-2000 global estimates (Children and adults), 2001 [http://w3.whosea.org/hivaids/fact.htm#End-2000%20global%20estimates]]. Most new infections have been acquired by the mucosal route, heterosexual transmission playing the major (≈80%) role. Although the incidence of transmission per unprotected coital act is estimated to be low (0.0001-0.004), but strikingly increased when acutely infected individuals are involved [Shattock, R. J., Moore, J. P.: Inhibiting sexual transmission of HIV-1 infection, Nat. Rev. Microbiol., (2003), 1:25-34; Pilcher, C. D., Tien, H., Eron, J. J., Vernazza, P. L., Leu, S-Y, Stewart, P. W., Goh, L-E, Cohen, M. S.: Brief but efficient: Acute HIV infection and the sexual transmission of HIV, J. Infect. Dis., (2004), 189:1785-1792], the cumulative effect is overwhelming.

Anti-HIV-1 vaccines applicable to global immunization programs are not expected to become available for many years. Thus, other prevention strategies are urgently needed. This includes educational efforts and application of mechanical and/or chemical barrier methods. The latter correspond to microbicides, i.e., topical formulations designed to block HIV-1 infection (and possibly transmission of other sexually transmitted diseases), when applied vaginally (and possibly rectally) before intercourse [Shattock, R. J., Moore, J. P.: Inhibiting sexual transmission of HIV-1 infection, Nat. Rev. Microbiol., (2003), 1:25-34; Stone, A.: Microbicides: A new approach to preventing HIV and other sexually transmitted infections, Nat. Rev. Drug Discov., (2002), 1:977-985; Shattock, R., Solomon, S.: Microbicides—aids to safer sex, Lancet, (2004), 363:1002-1003; Brown, H.: Marvellous microbicides. Intravaginal gels could save millions of lives, but first someone has to prove that they work, Lancet, (2004), 363:1042-1043]. Conceptually, it is preferred that the active ingredient(s) of microbicide formulations (1) block virus entry into susceptible cells by preventing HIV-1 binding to the cellular receptor CD4, the coreceptors CXCR4/CCR5 and to receptors on dendritic/migratory cells (capturing and transmitting virus to cells which are directly involved in virus replication), respectively [Shattock, R. J., Moore, J. P.: Inhibiting sexual transmission of HIV-1 infection, Nat. Rev. Microbiol., (2003), 1:25-34; Moore, J. P., Doms, R. W.: The entry of entry inhibitors: a fusion of science and medicine, Proc. Natl. Acad. Sci., USA, (2003), 100:10598-10602; Pierson, T. C., Doms, R. W.: HIV-1 entry inhibitors: new targets, novel therapies, Immunol. Lett., (2003), 85:113-118; Davis, C. W., Doms, R. W.: HIV Transmission: Closing all the Doors, J. Exp. Med., (2004), 199:1037-1040; Hu, Q., Frank, I., Williams, V., Santos, J. J., Watts, P., Griffin, G. E., Moore, J. P., Pope, M., Shattock, R. J.: Blockade of attachment and fusion receptors inhibits HIV-1 infection of human cervical tissue, J. Exp. Med., (2004), 199:1065-1075], and/or (2) are virucidal. The formulations must not adversely affect the target tissues, and should not cause them to become more susceptible to infection after microbicide removal [Fichorova, R. N., Tucker, L. D., Anderson, D. J.: The molecular basis of nonoxynol-9-induced vaginal inflammation and its possible relevance to human immunodeficiency virus type 1 transmission, J. Infect. Dis., (2001), 184:418-428; Fichorova, R. N., Bajpai, M., Chandra, N., Hsiu, J. G., Spangler, M., Ratnam, V., Doncel, G. F.: Interleukins (IL)-1, IL-6 and IL-8 predict mucosal toxicity of vaginal microbicidal contraceptives, Biol. Reprod. Epub ahead of print May 5 2004[http://www.biolreprod.org/cgi/rapidpdf/biolreprod.10 4.029603v1]].

Treatment with anti-retroviral drugs has decreased mortality from AIDS in industrialized countries, but has had a minimal effect so far in developing countries [Weiss, R.: AIDS: unbeatable 20 years on, Lancet, (2001), 357:2073-2074]. To avoid a similar dichotomy with respect to microbicides, they should be designed and selected to become affordable and widely accessible, while shortening the time between research and development and their marketing and distribution as much as possible. This would be facilitated if mass manufactured products with established safety records were to be found to have anti-HIV-1 activity. Qualifying candidates to be considered for microbicide development may possibly be discovered by screening pharmaceutical excipients (=“inactive” ingredients of pharmaceutical dosage forms) and foods, respectively, for anti-viral properties. This approach has already led to the discovery of cellulose acetate 1,2-benzenedicarboxylate (used for coating of enteric tablets and capsules) as a promising candidate microbicide [Neurath, A. R., Strick, N., Li, Y-Y, Lin, K., Jiang, S.: Design of a “microbicide” for prevention of sexually transmitted diseases using “inactive” pharmaceutical excipients, Biologicals, (1999), 27:11-21; Neurath, A. R., Strick, N., Li, Y-Y, Debnath, A. K.: Cellulose acetate phthalate, a common pharmaceutical excipient, inactivates HIV-1 and blocks the coreceptor binding site on the virus envelope glycoprotein gp120, BMC Infect. Dis., (2001), 1:17; Neurath, A. R., Strick, N., Jiang, S., Li, Y-Y, Debnath, A. K.: Anti-HIV-1 activity of cellulose acetate phthalate: Synergy with soluble CD4 and induction of “dead-end” gp41 six-helix bundles, BMC Infect. Dis., (2002), 2:6; Neurath, A. R., Strick, N., Li Y-Y: Anti-HIV-1 activity of anionic polymers: A comparative study of candidate microbicides, BMC Infect. Dis., (2002), 2:27; Neurath, A. R., Strick, N., Li, Y-Y: Water dispersible microbicidal cellulose acetate phthalate film, BMC Infect. Dis., (2003), 3:27]. The outcome of screening fruit juices neutralized to pH≈7 to discount nonspecific effects caused by acidity is described herein.

Pomegranates have been venerated for millennia for their medicinal properties and considered sacred by many of the world's major religions. In deference to pomegranates, the British Medical Association and several British Royal Colleges feature the pomegranate in their coat of arms. The Royal College of Physicians of London adopted the pomegranates in their coat of arms by the middle of the 16^{th} Century [Langley, P., Why a Pomegranate?, BMJ, 2000, 321:1153-1154]. The best known literary reference to the contraceptive power of pomegranate seeds is classical Greek mythology. Persephone had eaten six pomegranate kernels (from which juice is derived), while in the Underworld and for that many months the land remained infertile during the Fall and Winter. Ironically, this application shows that pomegranate juice contains HIV-1 or HIV-2 entry inhibitors corresponding to a class of anti-retroviral drugs still scarce in development [Greene, W. C., The brightening future of HIV therapeutics, Nat. Immunol., 2004, 5:867-871].

### SUMMARY OF THE INVENTION

It is an object of the present invention to provide a starch-pomegranate juice complex that can be used as a HIV-1 or HIV-2 entry inhibitor.

It is a further object of the present invention to prevent HIV-1 or HIV-2 infection.

These objects as well as other objects and advantages are provided by the present invention.

The present invention provides a complex comprising an active anti-HIV-1 or anti-HIV-2 ingredient of pomegranate juice that is adsorbed on a starch when the starch is in a water insoluble form. The complex inhibits HIV-1 or HIV-2 infection. The complex blocks the binding of HIV-1 or HIV-2 to the CD4 receptor and the CCR5 and CXCR4 coreceptors.

The present invention is also directed to a method of preventing HIV-1 or HIV-2 infection comprising administering to a mucous membrane of a human a pharmaceutically effective anti-HIV-1 or anti-HIV-2 amount of the above-described complex comprising an active anti-HIV-1 or anti-HIV-2 ingredient of pomegranate juice adsorbed on starch.

The present invention also relates to pharmaceutical compositions comprising a pharmaceutically effective anti-HIV-1 or anti-HIV-2 amount of the complex described above in combination with a pharmaceutically acceptable carrier.

The present invention also concerns a method of preventing HIV-1 or HIV-2 infection comprising administering to a mucous membrane of a human a pharmaceutically effective anti-HIV-1 or anti-HIV-2 amount of the pharmaceutical composition described above.

### BRIEF DESCRIPTION OF THE DRAWINGS

For the purpose of illustrating the invention, drawings are provided. It is to be understood, however, that the present invention is not limited to the precise subject matter depicted in the drawings.

FIG. 1 is a graph which depicts the inhibition of HIV-1 infection of HeLa-CD4-LTR-β-gal and U373-MAGI-CCR5E cells, respectively, by pomegranate juice (PJ).

The shaded area=HIV-1 IIIB; the unshaded area=HIV-1 BaL. Four distinct pomegranate juices (PJ1 to PJ4) were tested. Infection was monitored by measuring β-galactosidase.

FIG. 2 is a graph which depicts the inhibition of CD4 binding to recombinant gp120 IIIB and BaL, respectively, by pomegranate juice (PJ).

The wells were incubated with dilutions of the pomegranate juice for 1 hour at 37° C. After removal of the juice, and washing the wells, biotinyl-CD4 was added, and its binding to the wells was measured by ELISA.

FIG. 3 is a graph which depicts the inhibition by pomegranate juice (PJ) of binding to gp120 of antibodies to synthetic peptides from the gp120 sequence.

Wells of polystyrene plates coated with gp120 IIIB were incubated with 4-fold diluted pomegranate juice for 1 hour at 37° C. After removal of pomegranate juice, the wells were washed, and 50-fold diluted anti-peptide antisera [Neurath, A. R., Strick, N., Jiang, S.: Synthetic peptides and anti-peptide antibodies as probes to study interdomain interactions involved in virus assembly: The envelope of the human immunodeficiency virus (HIV-1), Virol., (1992), 188:1-13] were added. Bound IgG was quantitated by ELISA. Pomegranate juice was not added to control wells. Decreases of absorbance, as compared to the respective control wells, were plotted.

FIG. 4 is derived from the X-ray crystal structure and shows the location on the gp120 structure of segments corresponding to anti-peptide antibodies whose attachment to gp120 is inhibited by ≧50% in the presence of pomegranate juice (shaded area) and of amino acid residues involved in CD4 and CXCR4/CCR5 coreceptor binding, respectively.

The unshaded portions of the structure correspond to anti-peptide antibodies whose attachment to gp120 is not significantly inhibited by pomegranate juice.

The CD4 domains and the antigen-binding fragment of a neutralizing antibody were excised from the structure of the gp120-CD4-antibody complex [Kwong, P. D., Wyatt, R., Robinson, J., Sweet, R. W., Sodroski, J., Hendrickson, W. A.: Structure of an HIV gp120 envelope glycoprotein in complex with the CD4 receptor and a neutralizing human antibody, Nature, (1998), 393:648-659] (lgc1 retrieved from the Protein Data Bank (pdb) [http://www.rcsb.org/pdb/)]. The V3 loop, generated by homology modeling, was added to the gp120 structure as described [Neurath, A. R., Strick, N., Li, Y-Y, Debnath, A. K.: Cellulose acetate phthalate, a common pharmaceutical excipient, inactivates HIV-1 and blocks the coreceptor binding site on the virus envelope glycoprotein gp120, BMC Infect. Dis., (2001), 1:17]. The figure was generated by Molscript [Kraulis, P. J., MOLSCRIPT: a program to produce both detailed and schematic plots of protein structures, J. Appl. Cryst., (1991), 24:946-950] and Raster3D [Bacon, D. J., Anderson, W. F.: A fast algorithm for rendering space-filling molecule pictures, J. Mol. Graphics, (1988), 6:219-220; Merritt, E. A., Bacon, D. J.: Raster3D: Photorealistic molecular graphics, Methods Enzymol., (1997), 277:505-524]. The locations of gp120 variable loops (V1-V5) and of the N- and C-termini of the sequence are indicated.

FIG. 5 is a graph which shows the adsorption onto corn starch of gp120-CD4 binding inhibitor(s) from pomegranate juice (PJ).

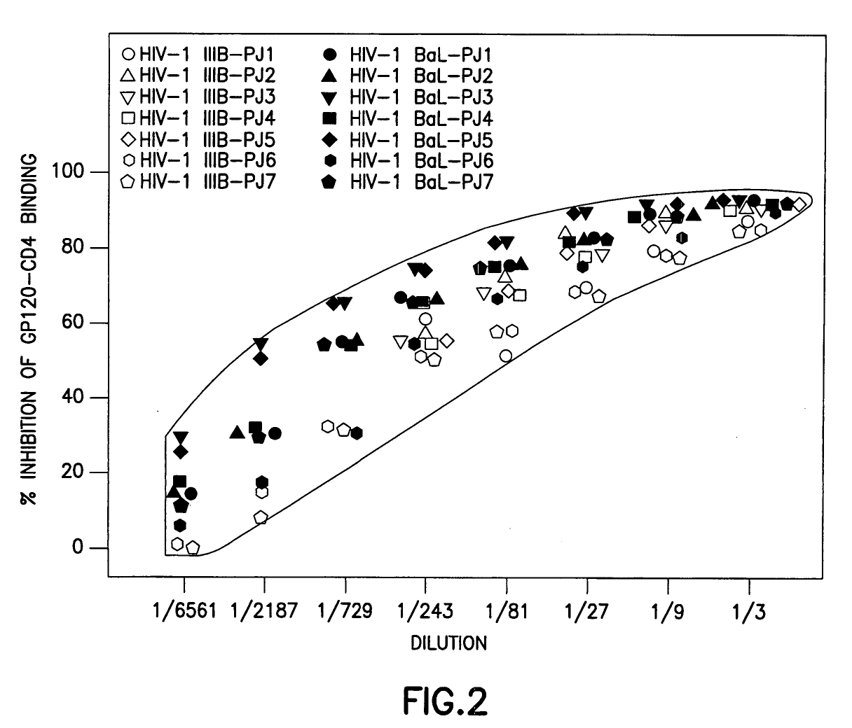

Corn starch (PURITY® 21, NF grade (S21); 200 mg/ml) was added to pomegranate juice prefiltered to remove particulates. After mixing for 1 hour at ≈20° C., the starch was allowed to settle and the supernatant fluid was removed by aspiration. The pellets, resuspended (200 mg/ml) in phosphate buffered saline, and the supernatant fluids were tested at serial dilutions for inhibition of CD4 binding to gp120 IIIB as described with respect to FIG. 2. The inhibitory activity of the resuspended pellet against gp120 BaL-CD4 binding was then confirmed. Control starch did not inhibit gp120-CD4 binding.

FIG. 6 is a graph which shows the inhibition by pomegranate juice (PJ) and PJ-S21, respectively, of gp120 IIIB-CD4 complex binding to cells expressing CXCR4 coreceptors.

HIV-1 IIIB gp120 (5 μg) and biotinyl-CD4 (2.5 μg) were added to 100 μl phosphate buffered saline (PBS) containing 100 μg/ml bovine serum albumin (BSA) (PBS-BSA) and pomegranate juice (PJ) (final 3-fold dilution) or PJ-S21 (67 mg). After 1 hour at 20° C., the respective mixtures were added to 10⁶ MT-2 cells. After 30 minutes, the cells were washed 3 times with PBS-BSA and PE-streptavidin (a fluorescent label specific for biotin; 0.1 μg) was added. After 20 minutes, the cells were washed and fixed by 1% formaldehyde in PBS. Flow cytometry analysis was performed in a FACSCalibur flow cytometer (Becton Dickinson Immunocytometric Systems, San Jose, Calif.) The median relative fluorescence values for cells exposed to gp120-CD4; gp120-CD4+PJ; gp120-CD4+PJ-S21; and control cells were: 13.7; 4.0; 4.3; and 2.1, respectively.

FIG. 7 is a graph which depicts the inhibition by pomegranate juice and PJ-S21, respectively of FLSC binding to CCR5 expressing Cf2Th/synCCR5 cells. FLSC is a chimeric recombinant protein consisting of gp120 BaL linked with D1D2 domains of CD4.

The inhibitory effect was quantitated using a cell-based ELISA [Zhao, Q., Alespeiti, G., Debnath, A. K.: A novel assay to identify entry inhibitorsthat block binding of HIV-1 gp120 to CCR5, Virol., 326:299-309]. The starting concentration of PJ-S21 was 200 mg/ml.

FIGS. 8A and 8B are graphs which depict the inhibition by PJ-S21 of biotinyl-gp 120 IIIB binding to peripheral blood mononuclear cells (PBMCs).

HIV-1 IIIB biotinyl gp 120 (5 μg) was added to 100 μl of PBS-BSA containing graded quantities of PJ-S21. After 1 hour at 20° C., the respective mixtures were added to 10⁶ PBMCs. After 30 minutes, the cells were washed 3 times with PBS-BSA and PE-streptavidin (0.1 μg was added). Subsequently, the procedures described above with respect to FIG. 6 were used. The median relative fluorescence values for control cells and cells exposed to biotinyl-gp 120 in the absence and presence of PJ-S21 (100, 6.25 and 3.12 mg/ml) were 4.1, 81.31, 12.2, 35.2 and 50.0, respectively. 100 mg of PJ-S21 corresponds to approximately 320 μg solids adsorbed from pomegranate juice onto starch.

FIG. 9 is a graph which shows that the inhibition of HIV-1 IIIB or BaL replication depends on the time of PJ-S21 addition pre-infection or post-infection.

For comparison, the inhibition of infection by the nonnucleoside reverse transcriptase inhibitor TMC-120, added to the cells at distinct intervals after HIV-1 was determined (dotted lines). Virus infection was measured by quantitation of β-galactosidase.

FIG. 10 is a graph which depicts the HIV-1 inhibitory and virucidal activity of PJ-S21 and its formulations.

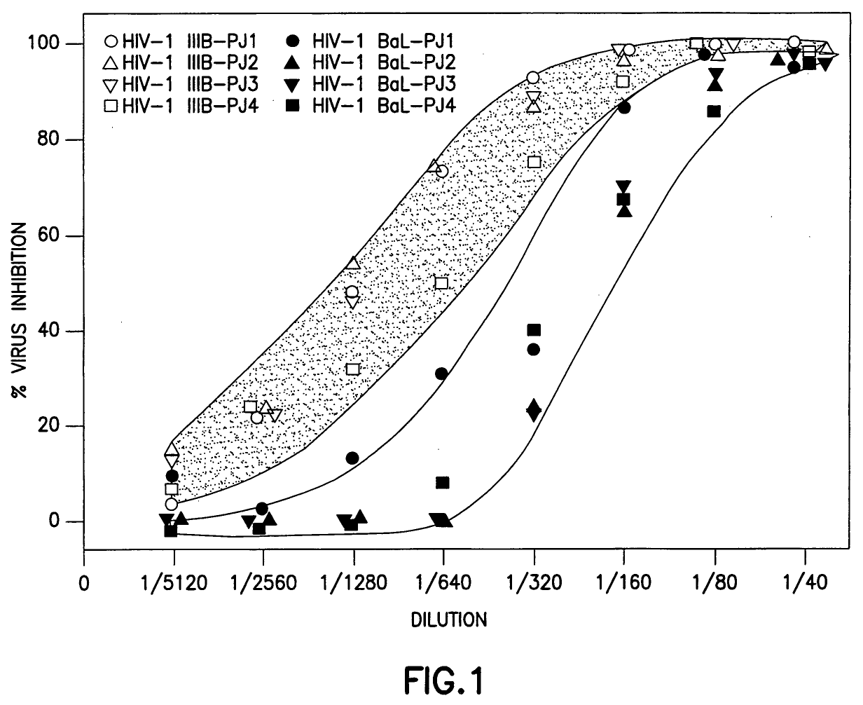

Inhibition of infection by HIV-1 IIIB and BaL, respectively, was determined as described with respect to FIG. 1. To measure virucidal activity, the respective viruses were mixed with graded quantities of PJ-S21 for 5 minutes at 37° C. After low speed centrifugation, the viruses were separated by precipitation with PEG 8000 and centrifugation. The resuspended pellets and control untreated viruses were serially diluted, and the dilutions assayed for infectivity. The concentration range given on the abscissa corresponds to 0.31 to 1,268 pg solids adsorbed from pomegranate juice to starch.

### DETAILED DESCRIPTION OF THE INVENTION

The complex according to the present invention comprises a starch and an active anti-HIV-1 or anti-HIV-2 ingredient of pomegranate juice that is adsorbed on the starch when the starch is in a water insoluble form. The starch is a starch which selectively adsorbs the active anti-HIV-1 or anti-HIV-2 ingredient of pomegranate juice.

The complex of the present invention inhibits HIV-1 or HIV-2 infection. The complex thus acts as a topical microbicide to block HIV-1 or HIV-2 infection. The complex is an HIV-1 or HIV-2 entry inhibitor (i.e., prevents entry of HIV-1 or HIV-2 into cells) since it blocks the binding of HIV-1 or HIV-2 to the CD4 receptor and the CCR5 and CXCR4 coreceptors.

The complex is produced by combining 100 to 250 mg of the starch with 1 ml of pomegranate juice, preferably by combining 150 to 225 mg of the starch with 1 ml of pomegranate juice.

The starch-pomegranate juice complex of the present invention can be administered to a mucous membrane of a man or a woman for preventing HIV-1 or HIV-2 infection. Thus, the starch-pomegranate juice complex can be applied to an internal body area, such as the vagina or rectum. Modes for administration include topically, vaginally or rectally.

The present invention is particularly effective for preventing HIV-1 or HIV-2 infection during sexual contact, such as sexual intercourse For administration to a human, it is preferred that the complex of the present invention be in a form of a pharmaceutical composition comprising a pharmaceutically effective anti-HIV-1 or anti-HIV-2 amount of the complex in combination with a pharmaceutically acceptable carrier.

The pharmaceutically acceptable carrier should preferably be such that the starch-pomegranate juice complex according to the present invention can be administered in the form of a suppository, a water dispersible film, a water dispersible tablet, sponge or a gel.

Pharmaceutical formulations suitable for rectal or vaginal administration, wherein the carrier is a solid, are most preferably represented as unit dose suppositories. Suitable carriers include a fatty acid suppository base (or a hydrogenated vegetable oil suppository base) known as “FATTIBASE,” cocoa butter and other materials commonly used in the art, and the suppositories may be conveniently formed by admixture of the complex of the present invention with the softened or melted carrier(s), followed by chilling and shaping in molds.

Mucoadhesive instantly dispersible (water dispersible) tablets can be prepared from the freeze-dried pomegranate/starch complex in combination with hydroxypropyl methylcellulose (“HPMC”), “PHARMABURST” (a quick dissolving delivery platform having a bulk density of 0.450, a tapped density of 0.536 and a Carr's index of 16.0% made by SPI Pharma), “CARBOPOL 947P” polymer (made by Noreon, Inc. of Cleveland, Ohio) and “CARBOPHIL” (made by Noreon, Inc. of Cleveland, Ohio).

The complex according to the present invention can be incorporated into a water dispersible film (similar to the widely available “breath control” strips).

The complex of the present invention can also be incorporated into a water dispersible sponge which is converted into a gel following topical application (see Neurath et al., BMC Infect. Dis., 2003, 3:27; U.S. Pat. No. 6,572,875; and U.S. Pat. No. 6,596,297 (the entire contents of U.S. Pat. No. 6,572,875 and U.S. Pat. No. 6,596,297 are hereby incorporated by reference herein).

The pharmaceutical compositions for use according to the present invention may also contain other active ingredients, such as spermicides, antimicrobial agents, preservatives or other anti-viral agents.

The pharmaceutical compositions of the present invention may also contain additives such as preservatives, flavors and fragrances. These additives may be present in any desired concentration. The concentrations of these additives will depend upon the desired properties, the agent to be released, the potency, the desired dosage, the dissolution times, etc.

Each of the above formulations should meet the following requirements: (1) minimization of waste disposal problems associated with the use of applicators needed for delivery of microbicidal gels/creams; (2) simplicity; (3) small packaging and discretion related to purchase, portability and storage; (4) low production costs; (5) amenability to industrial mass production at multiple sites globally and (6) potential application as rectal microbicides.

The complex can be administered in a concentration of 0.5 to 3 g, and preferably 1 to 1.5 g.

Furthermore, it would remain possible to produce for local use PJ-S21 based gel formulations with a limited shelf life, avoiding the costs of producing dry PJ-S21 powders via appropriate low temperature drying processes.

Whichever of these formulations is selected, adequate quality control will be needed to assure uniform anti-HIV-1 or anti-HIV-2 activity of the final product(s) and to establish reproducible conditions for manufacture.

In arriving at the present invention, fruit juices were screened for inhibitory activity against HIV-1 IIIB using CD4 and CXCR4 as cell receptors. The best juice was tested for inhibition of: (1) infection by HIV-1 BaL, utilizing CCR5 as the cellular coreceptor; and (2) binding of gp120 IIIB and gp120 BaL, respectively, to CXCR4 and CCR5. To remove most colored juice components, the adsorption of the effective ingredient(s) to dispersible excipients and other foods was investigated. A selected complex was assayed for inhibition of infection by primary HIV-1 isolates.

HIV-1 entry inhibitors from pomegranate juice were found to adsorb onto corn starch. The resulting complex blocks virus binding to CD4 and CXCR4/CCR5 and inhibits infection by primary virus clades A to G and group O.

The results herein demonstrate the feasibility of producing an anti-HIV-1 microbicide from inexpensive, widely available sources, whose safety has been established throughout centuries, provided that its quality is adequately standardized and monitored.

Pomegranate juice contains several ingredients [Poyrazoglu, E., Goekmen, V., Artik, N.: Organic acids and phenolic compounds in pomegranates (Punica granatum L.) Grown in Turkey, J. Food Composition and Analysis, (2002, 15:567-575; Module 2: Phytochemicals (minerals, phytamins, and vitamins)] which, isolated from natural products other than pomegranate juice, were reported to have anti-HIV activity, for example: caffeic acid [Mahmood, N., Moore, P. S., Detomassi, N., Desimone, F., Colman, S., Hay, A. J. P. C.: Inhibition of HIV-infection by caffeoylquinic acid-derivatives. Antivir. Chem. Chemother., (1993), 4:235-240], ursolic acid [Ma, C., Nakamura, N., Miyashiro, H., Hattori, M., Shimotohno, K.: Inhibitory effects of ursolic acid derivatives from cynomorium songaricum, and related triterpenes on human immunodeficiency viral protease, Phytotherapy Research, (1998), 12:s138-142], catechin and quercetin [Mahmood, N., Piacente, S., Pizza, C., Burke, A., Khan, A. I., Hay, A. J.: The anti-HIV activity and mechanisms of action of pure compounds isolated from, Rosa damascena, Biochem. Biophys. Res. Commun., (1996), 229:73-79; DeTommasi, N., Piacente, S., Rastrelli, L., Mahmood, N., Pizza, C.: Anti-HIV activity directed fractionation of the extracts of Margyricarpus setosus, Pharmaceutical Biology, (1998), 36:29-32]. However, these compounds, in purified form, obtained commercially, did not block (at 200 μg/ml) gp120-CD4 binding as measured by the ELISA described herein and did not adsorb to corn starch, unlike the entry inhibitor(s) from pomegranate juice. In fact, the supernatant after treatment of pomegranate juice with starch, and removal of the entry inhibitors, retained anti-HIV-1 activity and also inhibited infection by herpes virus type 1, unlike the HIV-1 entry inhibitors which adsorbed onto starch. Thus, the antiviral activities in the supernatant appeared to be non-specific, and probably similar to those of extracts from pomegranate rind [Reuters NewMedia, Inc: Pomegranates could help in battle against AIDS, 1996 Mar. 10. http://www.aegis.com/news/re/1996/RE960310.html; British Muslims Monthly Survey: Medical breakthrough. 1996 March; IV:6 [http://artsweb.bham.ac.uk/bmms/1996/03March96.html#Medical%20breakthrough], and were not characterized further.

Additional information [Jassim, S. A. A., Denyer, S. P., and Stewart, G. S. A. B.: Antiviral or antifungal composition comprising an extract of pomegranate rind or other plants and method of use, U.S. Pat. No. 5,840,308 issued Nov. 24, 1998; Shehadeh, A. A.: Herbal extract composition and method with immune-boosting capability,. U.S. Pat. No. 6,030,622 issued Feb. 29, 2000; Jassim, S. A. A., Denyer, S. P., and Stewart, G. S. A. B.: Antiviral or antifungal composition and method, U.S. Pat. No. 6,187,316 issued Feb. 2, 2001; Jassim, S. A. A. and Denyer, S. P.: Antiviral or antifungal composition and method, U.S. patent application 2002/0064567 published May 30, 2002], has revealed that the findings apply to crude extracts from pomegranate rind prepared at elevated temperatures under conditions which destroy the HIV-1 entry inhibitor described herein.

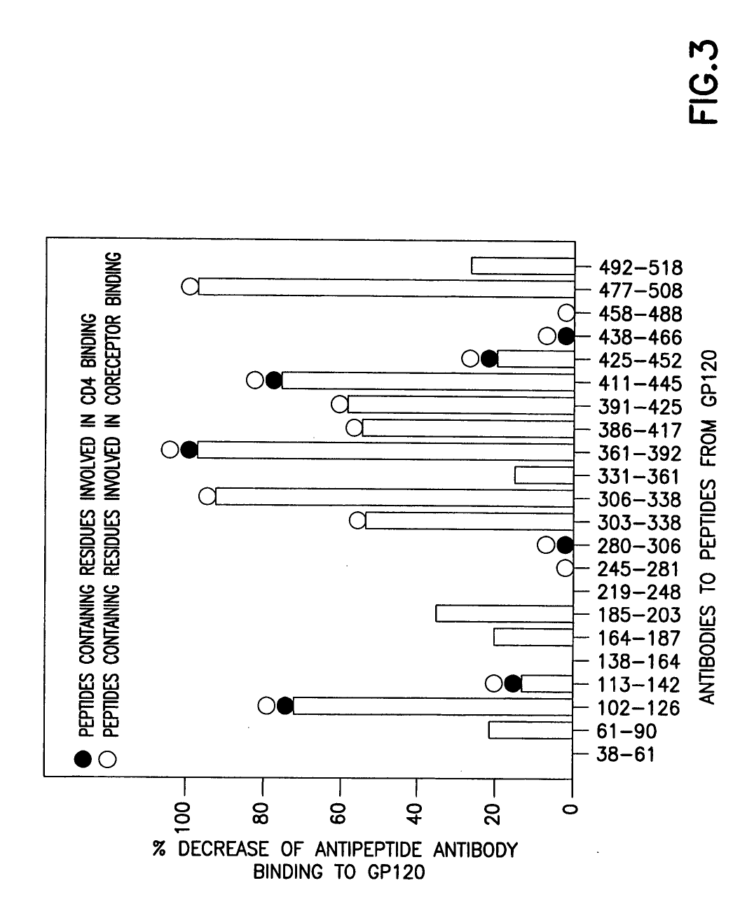

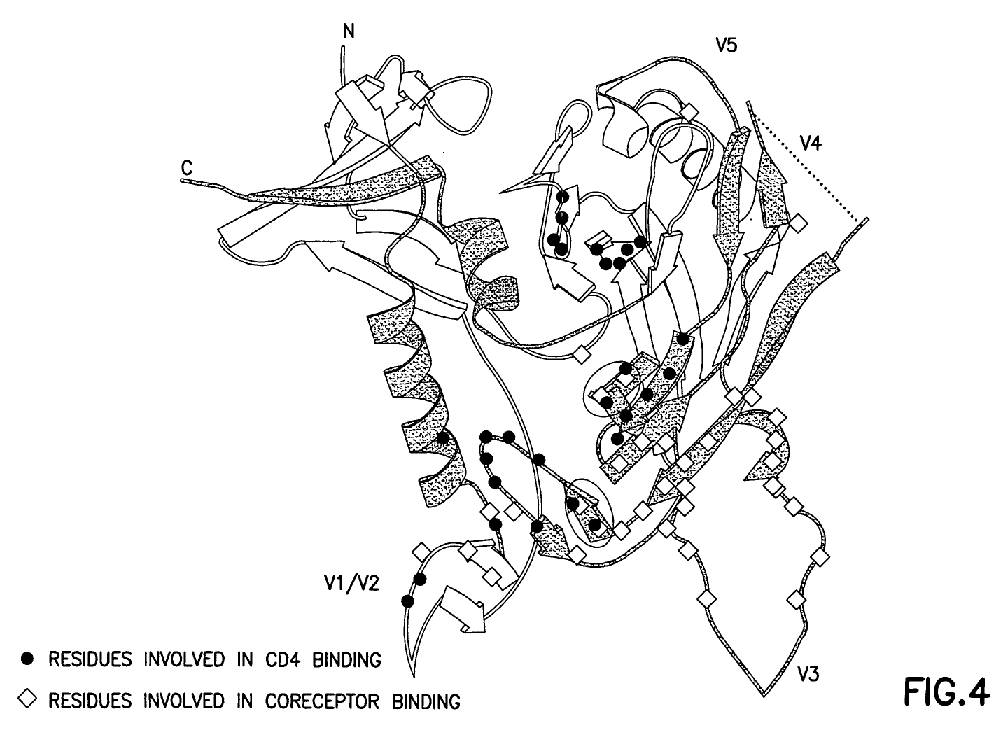

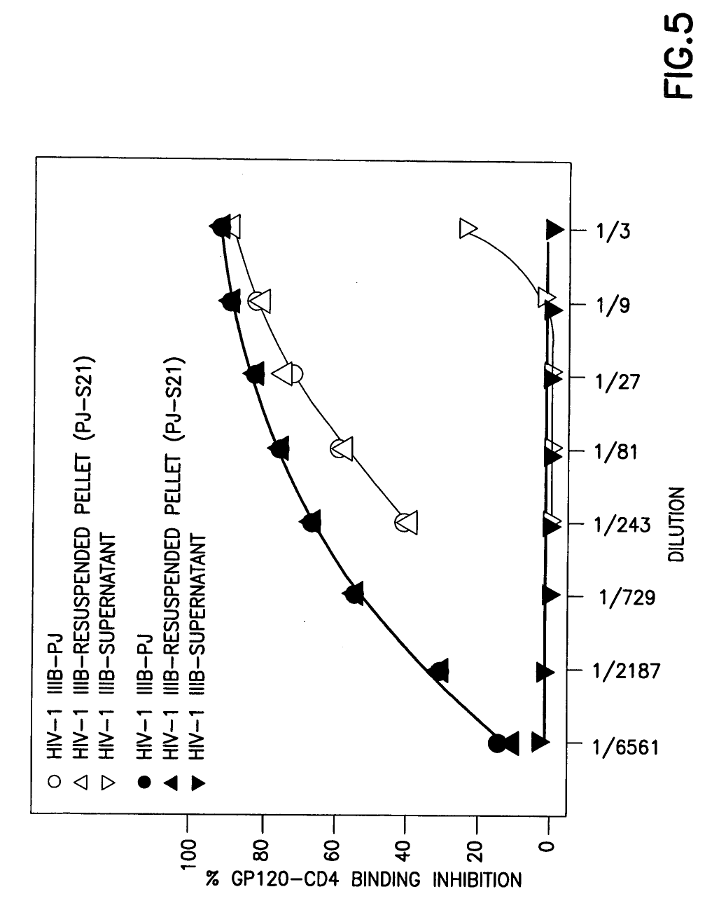

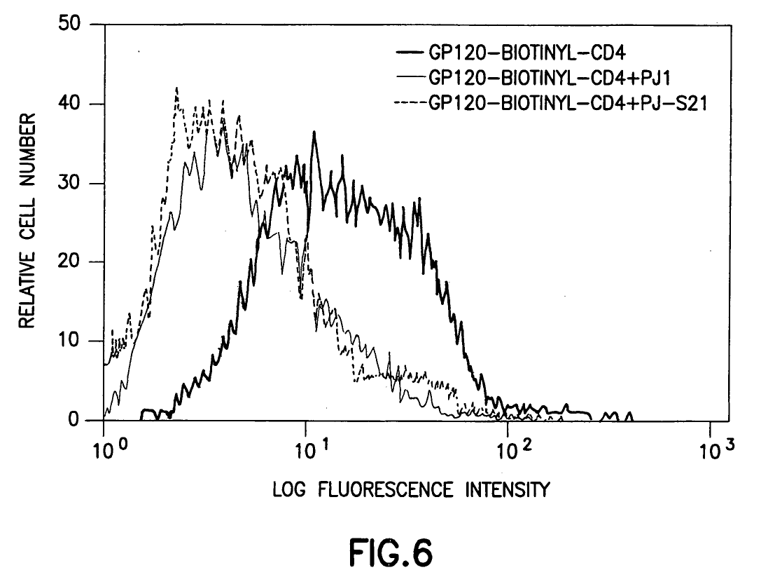

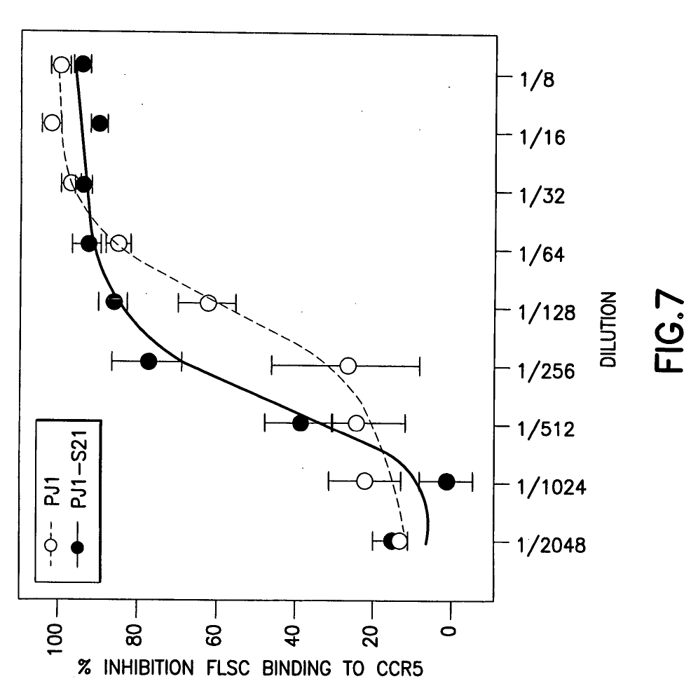

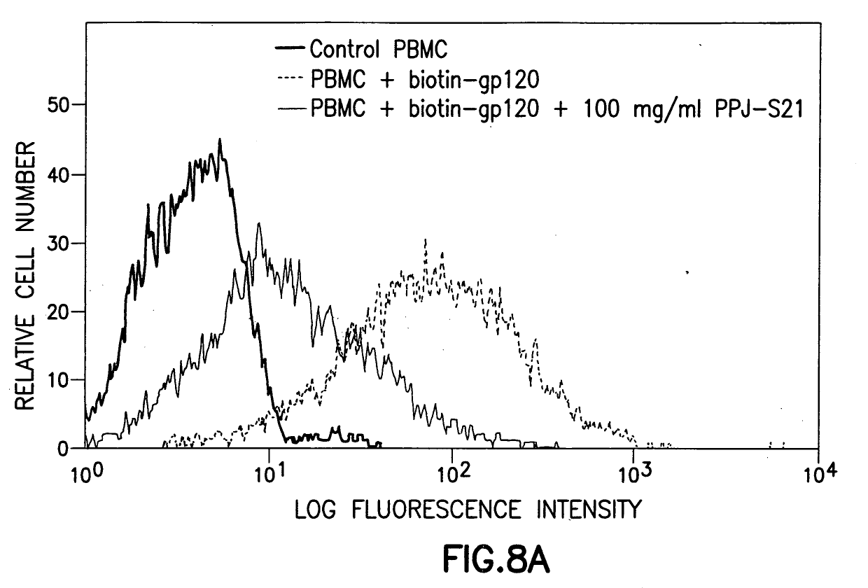

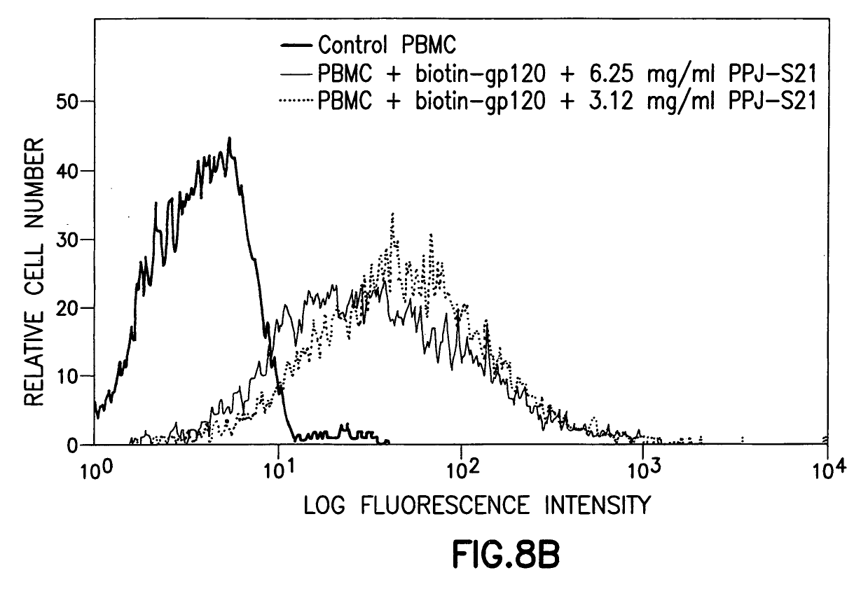

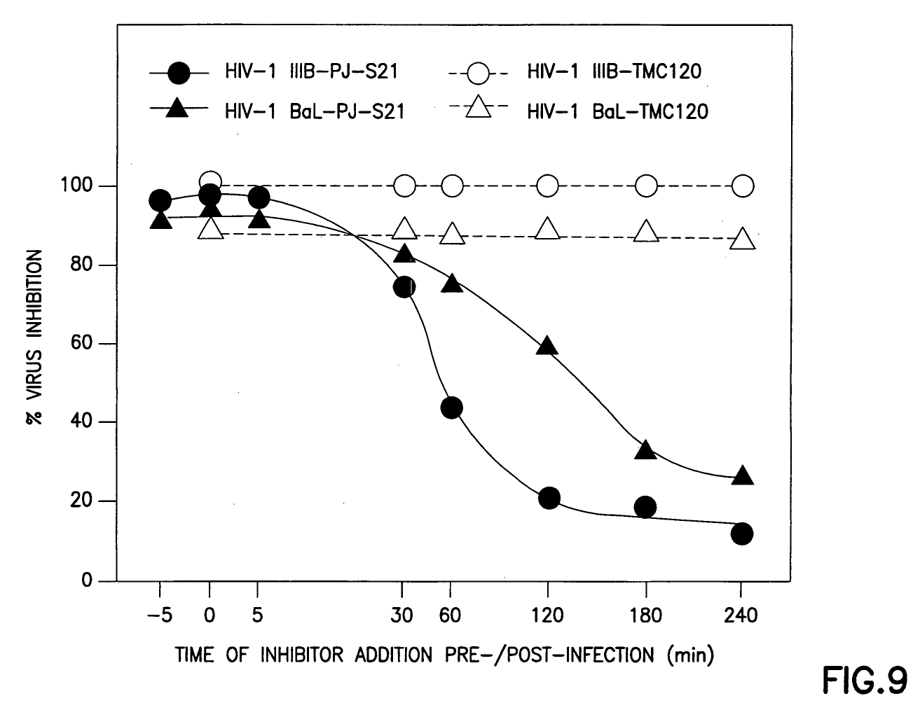

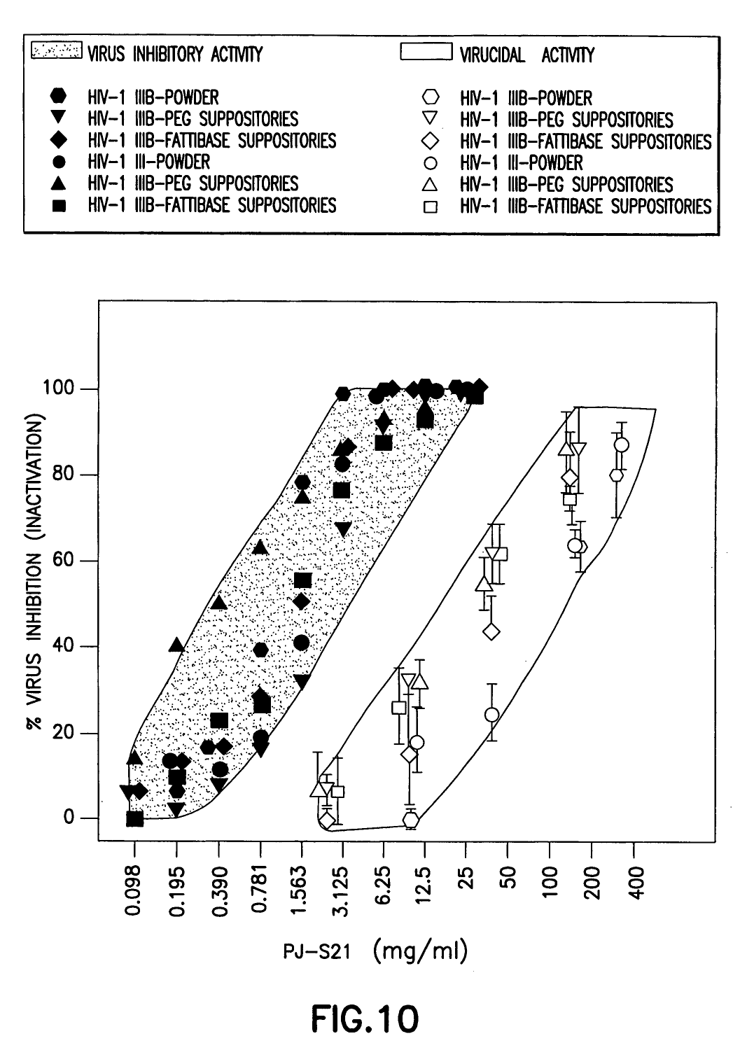

The inhibitor(s) interfering with gp120 binding to CD4 (FIGS. 2 and 5) blocked additional sites on gp120 (FIG. 3) involved in interaction with the CXCR4/CCR5 coreceptors (FIGS. 4, 6 and 7). This was not completely expected and can be explained either by the presence of multiple inhibitors with distinct or overlapping specificities in PJ-S21 or by induction of gp120 conformational changes [Hsu, S-T, Bonvin, A. M. J. J.: Atomic insight into the CD4 binding-induced conformational changes in HIV-1 gp120, Proteins, (2004), 55:582-593] resulting in blockade of both CD4 and CXCR4/CCR5 binding sites on gp120. Similar effects have been noticed for other small molecule inhibitors [Neurath, A. R., Strick, N., Lin, K., Debnath, A. K., Jiang, S.: Tin protoporphyrin IX used in control of heme metabolism in humans effectively inhibits HIV-1 infection, Antiviral Chem. Chemother., (1994), 5:322-330]. Simultaneous blocking of more than a single site on HIV-1 involved in virus entry is expected to increase the effectiveness of candidate microbicides [Hu, Q., Frank, I., Williams, V., Santos, J. J., Watts, P., Griffin, G. E., Moore, J. P., Pope, M., Shattock, R. J.: Blockade of attachment and fusion receptors inhibits HIV-1 infection of human cervical tissue, J. Exp. Med., (2004), 199:1065-1075]. The target sites for the inhibitor(s) are likely to be located within the protein moiety of gp120 since binding of labeled Galanthus nivalis lectin (specific for terminal mannose residues) [Hammar, L., Hirsch, I., Machado, A. A., de Mareuil, J., Baillon, J. G., Bolmont, C., Chermann, J-C: Lectin-mediated effects of HIV type 1 infection in vitro, AIDS Res. Hum. Retroviruses, (1995), 11:87-95]; and other lectins to gp120 oligosaccharides was not diminished in the presence of pomegranate juice or PJ-S21 (data not shown).

Blocking of CD4 binding sites on HIV-1 gp120 by monoclonal antibodies or a CD4-IgG2 recombinant protein has been shown to be sufficient to inhibit HIV-1 infection of human cervical tissue ex vivo [Hu, Q., Frank, I., Williams, V., Santos, J. J., Watts, P., Griffin, G. E., Moore, J. P., Pope, M., Shattock, R. J.: Blockage of attachment and fusion receptors inhibits HIV-1 infection of human cervical tissue, J. Exp. Med., 2004, 199:1065-1075] and in preventing virus transmission to macaque monkeys when applied vaginally [Veazey, R. S., Shattock, R. J., Pope, M., Kirijan, J. C., Jones, J., Hu, Q., Ketas, T., Marx, P. A., Klasse, P. J., Burton, D. R., Moore, J. P.: Prevention of virus transmission to macaque moneys by a vaginally applied monoclonal antibody to HIV-1 gp120, Nat. Med., 2003, 9:343-346]. Therefore, it is expected that PJ-S21 will be similarly effective.

The application of PJ-S21 as a topical anti-HIV-1 microbicide requires reasonable uniformity among batches produced at distinct times and locations. Similarities in gp120-CD4 binding inhibitory activity among distinct freshly prepared and commercial juices stored for unknown periods (FIG. 2) suggest that this should be feasible. Pasteurization of juice for 30 seconds at 85° C. resulted in complete loss of inhibitory activity. A commercial pomegranate juice concentrate exposed to 61° C., and two other concentrates, presumably prepared by evaporation at elevated temperatures, had no or drastically diminished activity. The gp120-CD4 inhibitory activity from PJ3 (juice with fructose and citric acid added), failed to bind to starch. Separate experiments revealed that these compounds interfere with inhibitor binding to corn starch. Therefore, pomegranate juices intended for production of the PJ-S21 complex must be sterilized by filtration and be free of additives.

Particular attention must be devoted to the selection of starch, a pharmaceutical excipient generally used in vaginal formulations [Garg, S., Tambweker, K. R., Vermani, K., Garg, A., Kaul, C. L., Zaneveld, L. J. D.: Compendium of pharmaceutical excipients for vaginal formulations, Pharmaceutical Technoloqy Drug Delivery, (2001), September:14-24. [http://ptech.adv100.com/pharmtech /data/articlestandard/pharmtech/512001/5133/article.pdf]]for effective binding of the virus entry inhibitors from pomegranate juice. Among a dozen starches tested, the best results were obtained with PURITY® 21 corn starch NF grade (S21). However, it is considered that different brands of starch may be effective, and this invention is not limited to S21. With other brands of starch that were tested, the adsorption of the inhibitors was either incomplete or their binding did not result in a complex having activity in the ELISA measuring gp120-CD4 binding inhibition (ARGO® corn starch), presumably, because of irreversible binding of the pomegranate juice inhibitors.

Interestingly, there are only a few references available regarding the use of starch as an adsorbent for different compounds: flavors [Yao, W., Yao, H.: Adsorbent characteristics of porous starch, Starch/Starke, 2002, 54:260-263; Whistler, R. L.: Microporous granular starch matrix compositions, U.S. Pat. No. 4,985,082 issued Jan. 15, 1991], dyes [Berset, C., Clermont, H., Cheval, S.: Natural red colorant effectiveness as influenced by absorptive supports, J. Food Sci., 1995, 60:858-861, 879; Stute, R., Woelk, H. U.: Interaction between starch and reactive dyes, New technique for the investigation of starch. II. Influence of fixation reaction of starch, Starch/Starke, 1974, 26:1-9; Seguchi, M.: Dye binding to the surface of wheat starch granules, Cereal Chemistry, 1986, 63:518-520], low-molecular mass saccharides [Tomasik, P., Wang, Y-J, Jane, J. L.: Complexes of starch with low-molecular saccharides, Starch/Starke, 1995, 47:185-191], lipids [Zhang, G., Maladen, M. D., Hamaker, B. R.: Detection of novel three component complex consisting of starch, protein, and free fatty acids, J. Agric. Food Chem., 2003, 51:2801-2805; Johnson, J. M., Davis, E. A., Gordon, J.: Lipid binding of modified corn starches studies by electron spin resonance, Cereal Chemistry, 1990, 67:236-240], proteins [Tomazic-Jezic, V. J., Lucas, A. D., Sanchez, B. A.: Binding and measuring natural rubber latex proteins on glove powder, J. Immunoassay Immunochem., 2004, 25:109-123] and iodine [Conde-Petit, B., Nuessli, J., Handshin, S., Escher, F.: Comparative characterization of aqueous starch dispersions by light microscopy, rheometry, and iodine binding behavior, Starch/Starke, 1998, 50:184-192].

The intended dose of the complex of the invention, for example, PJ-S21 for vaginal application is 1.0 to 1.5 g (=3.17-4.76 mg solids from pomegranate juice adsorbed onto starch), i.e., ≧100-fold higher than the dose needed for blocking HIV-1 infection in vitro (FIG. 10, Table 1), and thus expected to meet requirements for likely in vivo protection against vaginal challenge [Moore, J., Wainberg, M., Amman, A., Veazey, R., Pope, M., Shattock, R. J., Doms, R. W.: Development of fusion/entry inhibitors as topical microbicides, In Proceeding of the Microbicides, (2004): 28-31 Mar. 2004; London [http://www.microbicides2004.org.uk/progtue. html]]. This quantity of PJ-S21 is produced from 5 to 7.5 ml of pomegranate juice, i.e., ≦5% of a single (150 ml) serving of juice, attesting to the-safety, feasibility and economy of this proposed candidate topical microbicide.

### EXAMPLES

The present invention will now be described with reference to the following non-limiting examples.

Reagents

Pomegranate juices (“PJs”) were purchased in local Stores in New York, N.Y., U.S.A.; their origin is Given in parentheses: PJ1 (Madeira Enterprises Inc., Madeira, Calif.); PJ2 was prepared from fresh ripe Pomegranates in the laboratory of the inventors; PJ3(Sadaf7; Sadaf7 Foods, Los Angeles, Calif.; additional ingredients: fructose, citric acid); PJ4 (Cortas Canning & Refrigeration Co. S.A.L., Beirut, Lebanon); PJ5 (Kradjian, Import & Wholesale Distribution, Glendale, Calif., Product of Iran); PJ6 (R. W. Knudson; Just Pomegranate; Knudsen & Sons, Inc., Chico, Calif.); PJ7 (Aromaproduct Ltd., Product of Georgia; distributed by Tamani, Inc., New York, N.Y.). Starches used were as follows: PURE-DENT® B815 Corn Starch NF, PURE-DENT® B816 Corn Starch USP, Spress® B825 Pregelatinized corn starch NF, Spress® B820 Pregelatinized corn starch NF, INSTANT PURE-COTE™ B792 Food starch-modified, INSCOSITY™ B656 Food starch-modified (Grain Processing Corporation, Muscatine, Ind.); PURITY® 21 corn starch NF and PURITY® 826 corn starch NF (National Starch and Chemical Company, Bridgewater, N.J.); Remyline AX-DR Waxy rice starch and Remy DR native rice starch, medium grind (A&B Ingredients, Fairfield, N.J.); ARGO® corn starch (Best Foods Division, CPC International Inc., Engelwood Cliffs, N.J.); STALEY® pure food powder starch (Tate & Lyle, Decatur, Ill.); STARCH 1500 pregelatinized starch NF (Colorcon, West Point, Pa.).

The following polymers were used: polyethylene glycols (PEG) 1000 NF, 1500 NF and 8000 NF; and hydroxypropyl methylcellulose (HPMC), 50 cps, USP (Spectrum, New Brunswick, N.J.); Carbopol 974P-NF (B. F. Goodrich Co., Cleveland, Ohio); Carbophil, Noveon AA1 (Noveon, Inc., Cleveland Ohio); and Pharmaburst B2 (SPI Pharma, New Castle, Del.). Fattibase was from Paddock Laboratories, Inc., Minneapolis, Minn.

The following recombinant proteins were employed: HIV-1 IIIB gp120, biotinyl-HIV-1 IIIB gp120, CD4, and biotinyl-CD4 (ImmunoDiagnostics, Inc., Woburn, Mass.); HIV-1 IIIB BaL gp120 and FLSC (a full length single chain protein consisting of BaL gp120 linked with the D1D2 domains of CD4 by a 20 amino acid linker) (produced in transfected 293T cells [Zhao, Q., Alespeiti, G., Debnath, A. K.: A novel assay to identify entry inhibitors that block binding of HIV-1 gp120 to CCR5, Virol., 326:299-309].

Phycoerythrin (PE)-labeled streptavidin was from R & D Systems, Minneapolis, Minn. Biotinylated Galanthus nivalis lectin was from EY Laboratories, Inc., San Mateo, Calif.

Rabbit antibodies to synthetic peptides from gp120 (residue numbering as in Neurath, A. R., Strick, N., Jiang, S.: Synthetic peptides and anti-peptide antibodies as probes to study interdomain interactions involved in virus assembly: The envelope of the human immunodeficiency virus (HIV-1), Virol. (1992), 188:1-13) were prepared as described in Neurath et al., Virol., (1992), 188:1-13.

Monoclonal antibodies (mAb) 588D, specific for the CD4 binding site, and 9284, specific for the gp120 V3 loop, were from Dr. S. Zolla-Pazner and NEN Research Products, Du Pont, Boston, Mass., respectively. A “generic” version of the nonnucleoside HIV-1 reverse transcriptase inhibitor TMC-120 [Van Herrewege, Y., Michiels, J., Van Roey, J., Fransen, K., Kestens, L., Balzarini, J., Lewi, P., Vanham, G., Janssen, P.: In vitro evaluation of nonnucleoside reverse transcriptase inhibitors UC-781 and TMC120-R147681 as human immunodeficiency virus microbicides, Antimicrob. Agents Chemother., (2004), 48:337-339] was synthesized by Albany Molecular Research, Inc., Albany, N.Y., and used in control experiments at a final 5 μM concentration.

Pelletted, 1000-fold concentrates of HIV-1 IIIB (6.8×10¹⁰ virus particles/ml) and BaL (2.47×10¹⁰ virus particles/ml) were from Advanced Biotechnologies, Inc., Columbia, Md.

Primary HIV-1 isolates, MT-2 cells, HeLa-CD4-LTR-β-gal and U373-MAGI-CCR5E cells and Cf2Th/synCCR5 cells were obtained from the AIDS Research and Reference Reagent Program operated by McKesson BioServices Corporation, Rockville, Md.

CEMx174 5.25M7 cells, transduced with an HIV-1 long terminal repeat (LTR)-green fluorescent protein and luciferase reporter construct, expressing CD4 and CXCR4 and CCR5 coreceptors [Hsu, M., Harouse, J. M., Gettie, A., Buckner, C., Blanchard, J., Cheng-Mayer, C.: Increased mucosal transmission but not enhanced pathogenicity of the CCR5-tropic, simian AIDS-inducing simian/human immunodeficiency virus SHIV_{SF162P3} maps to envelope gp120, J. Virol., (2003), 77:989-998], were obtained from Dr. Cecilia Cheng-Mayer. The cells were maintained in RPMI-1640 medium supplemented with 10% fetal bovine serum (FBS), 1 μg/ml puromycin and 200 μg/ml G418. These cells are suitable for titration of both X4 and R5 HIV-1 isolates and for determining the effectiveness of anti-HIV-1 drugs with reliable reproducibility. This is impossible to accomplish by using peripheral blood mononuclear cells (PBMCs) because of their variations in susceptibility to HIV-1 infection among cells derived from distinct individuals [Schwartz, D. H., Castillo, R. C., Arango-Jaramillo, S., Sharma, U. K., Song, H. F., Sridharan, G.: Chemokine-independent in vitro resistance to human immunodeficiency virus (HIV-1) correlating with low viremia in long-term and recently infected HIV-1-positive persons, J. Infect. Dis., 1997, 176:1168-1174; Wu, L., Paxton, W. A., Kassam, N., Ruffing, N., Rottman, J. B., Sullivan, N., Choe, H., Sodroski, J., Newman, W., Koup, R. A., Mackay, C. R.: CCR5 levels and expression pattern correlate with infectability by macrophage-tropic HIV-1, in vitro, J. Exp. Med., 1997, 185:1681-1691; Blaak, H., Ran, L. J., Rientsma, R., Schuitmaker, H.: Susceptibility of in vitro stimulated PBMC to infections with NS1 HIV-1 is associated with levels of CCR5 expression and beta-chemokine production, Virol., 2000, 167:237-246]. PBMCs were isolated from HIV-1 negative donors as described [Gartner, S., Popovic, M.: Virus isolation and production, In Techniques in HIV Research, Edited by Aldovini, A., Walker, B. D., New York; M., Stockton Press; 1990:53-70].

Formulations

In attempts to separate gp120-CD4 binding inhibitory activity from most other ingredients of pomegranate juice, 200 mg of distinct starch preparations were added per ml of pomegranate juice. After mixing for 1 hour at 20° C., excess juice was decanted, and the pellets resuspended in 1 ml of distilled water. Based on results of enzyme linked immunosorbent assays (ELISA), PURITY® 21 corn starch, NF grade (S21) was selected for further studies, and the corresponding pomegranate juice complex was designated as PJ-S21. PJ-S21 was freeze-dried and used to prepare the following formulations: PEG suppositories (50% PJ-S21, 45% PEG 1000, 5% PEG 1500); Fattibase suppositories (50% pomegranate juice-S21, 50% Fattibase); and mucoadhesive instantly dispersible tablets (50% PJ -S21, 20% HPMC, 20% Pharmaburst, 7.5% Carbopol 974P and 2.5% Carbophil).

Enzyme Linked Immunosorbent Assays (ELISA)

Inhibition of infection by HIV-1 IIIB and BaL, respectively, was determined relying on a β-galactosidase readout system [Neurath, A. R., Strick, N., Li, Y-Y: Anti-HIV-1 activity of anionic polymers: A comparative study of candidate microbicides, BMC Infect. Dis., (2002), 2:27]. The enzyme was quantitated with a Galacto-Light Plus System chemiluminescence reporter assay (Applied Biosystems, Foster City, Calif.) using a Microlight ML 2250 luminometer (Dynatech Laboratories, Inc., Chantilly, Va.). To measure virucidal activity, virus was separated from excess inactivating agent by centrifugation and/or precipitation with PEG 8000 [Neurath, A. R., Strick, N., Li, Y-Y: Anti-HIV-1 activity of anionic polymers: A comparative study of candidate microbicides, BMC Infect. Dis. (2002), 2:27; Neurath, A. R., Strick, N., Li, Y-Y: Water dispersible microbicidal cellulose acetate phthalate film, BMC Infect. Dis. (2003), 3:27]. Serial dilutions of the treated virus were assayed for infectivity as described above. Dose response curves (i.e., luminescence vs. dilution) for treated and control viruses were obtained, and the percentages of virus inactivation were calculated [Neurath, A. R., Strick, N., Li, Y-Y: Water dispersible microbicidal cellulose acetate phthalate film, BMC Infect. Dis., (2003), 3:27]. To determine inhibition of infection by primary HIV-1 strains, CEMx174 5.25 M7 cells were incubated with 100x TCID₅₀ of primary HIV-1 strains in the absence or presence of PJ-S21 at graded concentrations for 3 days at 37° C. The experiments were done in triplicate. Infection was quantitated by measuring luciferase activity [Hsu, M., Harouse, J. M., Gettie, A., Buckner, C., Blanchard, J., Cheng-Mayer, C.: Increased mucosal transmission but not enhanced pathogenicity of the CCR5-tropic, simian AIDS-inducing simian/human immunodeficiency virus SHIV_{SF162P3} maps to envelope gp120, J. Virol., (2003), 77:989-998] using a kit from Promega (Madison, Wis.) in an Ultra 384 luminometer (Tecan, Research Triangle Park, N.C.).

CD4-HIV-1 gp120 binding and its inhibition were measured by ELISA. Wells of 96-well polystyrene plates (Immulon II, Dynatech Laboratories, Inc., Chantilly, Va.) were coated with 100 ng/well of either gp120 IIIB or gp120 BaL, and post-coated as described [Neurath, A. R., Strick, N., Li, Y. Y., Debnath, A. K.: Cellulose acetate phthalate, a common pharmaceutical excipient, inactivates HIV-1 and blocks the coreceptor binding site on the virus envelope glycoprotein gp120, BMC Infect. Dis., (2001), 1:17]. Dilutions of pomegranate juices and of PJ-S21, respectively, in 0.14 M NaCl, 0.01 M Tris, 0.02% sodium merthiolate, pH 7.0 (TS) containing 100 μg/ml bovine serum albumin (BSA) were added to the wells for 1 hour at 37° C. The wells were washed 5× with TS. Biotinyl-CD4 (1 μg/ml) in TS-1% gelatin was added to the wells for 5 hours at 37° C. After washing 1× with TS-0.1% Tween 20 and 5× with TS, horseradish peroxidase (HRP)-streptavidin (0.625 μg/ml; Amersham, Arlington Heights, Ill.) in TS-2% gelatin-0.05% Tween 20 was added. After 30 minutes at 37° C., the wells were washed 4 x with TS-0.1% Tween 20 and 2× with TS. Bound HRP-was detected using a kit from Kirkegaard and Perry Laboratories Inc. (Gaithersburg, Md.) and the absorbance (A) read at 450 nm. A in the absence of inhibitors was 1.0 to 1.5, and 0 to 0.005 in the absence of biotinyl-CD4. In an alternative assay, CD4 (500 ng/ml) was mixed with biotinyl-gp120 (1 μg/ml) in the presence or absence of inhibitors for 30 minutes at 20° C. Serial dilutions of the mixtures were added to wells coated with the anti-CD4 mAb OKT 4 (Ortho-Clinical Diagnostics, Rochester, N.Y.) and captured biotinyl-gp120 was detected with HRP-streptavidin as described above. To measure binding to gp120 of antibodies to gp120 peptides, the respective rabbit antisera were diluted 50-fold in a mixture of FBS and goat serum (9:1) containing 0.1% Tween 20 (pH 8.0) and added to gp120 wells. Bound IgG was detected with HRP labeled anti-rabbit IgG (Sigma, St. Louis, Mo.; 1 μg/ml in TS-10% goat serum-0.1% Tween 20). A cell-based ELISA was used to measure the blocking of CCR5 binding sites on HIV-1 BaL gp120 by PJ and PJ-S21, respectively [Zhao, Q., Alespeiti, G., Debnath, A. K.: A novel assay to identify entry inhibitors that block binding of HIV-1 gp120 to CCR5, Virol., 326:299-309]. Briefly, FLSC (125 ng/ml) in the absence or presence of graded amounts of inhibitors was added to Cf2Th/synCCR5 cells fixed with 5% formaldehyde in wells of 96-well plates. After 1 hour at 37° C., bound FLSC was detected with mAb M-T441 (125 ng/ml; Ancell, Bayport, Minn.) specific for the CD4 D2 domain, followed sequentially by biotinylated anti-mouse IgG and HRP-streptavidin.

Results

Anti-HIV-1 Activity of Pomegranate Juice

Serial twofold dilutions of juices [apple, black cherry, blueberry, coconut milk, cranberry, elderberry, grape (red), grapefruit, honey, lemon, lime, pineapple, pomegranate and red beet (10% reconstituted dry powder)] were assayed for inhibition of infection by HIV-1 IIIB of cells expressing the CD4 and CXCR4 receptors and coreceptors. Most juices (4-fold diluted) had no inhibitory activity, except blueberry, cranberry, grape and lime juice, respectively [endpoints for 50% inhibition of infection (ED₅₀) between 1/16 and 1/64]. Consistently, pomegranate juices from distinct geographical areas had the highest inhibitory activity (FIG. 1; shaded area). Since HIV-1 viruses utilizing CCR5 as a coreceptor (=R5 viruses) are predominantly transmitted sexually [Shattock, R. J., Moore, J. P.: Inhibiting sexual transmission of HIV-1 infection, Nat. Rev. Microbiol., (2003), 1:25-34; Shattock, R. J., Doms, R. W.: AIDS models: Microbicides could learn from vaccines, Nat. Med., (2002), 8:425], it was important to test whether pomegranate juice can inhibit not only infection by HIV-1 IIIB, a virus utilizing CXCR4 as a coreceptor (=X4 virus), but also infection by a R5 virus, HIV-1 BaL. The results in FIG. 1 (unshaded area) show that infection by the latter virus is also inhibited, albeit less effectively than that by HIV-1 IIIB.

Blocking virus entry is a primary target for microbicide development [Shattock, R. J., Moore, J. P.: Inhibiting sexual transmission of HIV-1 infection, Nat. Rev. Microbiol., (2003), 1:25-34; Moore, J. P., Doms, R. W.: The entry of entry inhibitors: a fusion of science and medicine, Proc. Natl. Acad. Sci. USA, (2003), 100:10598-10602; Pierson, T. C., Doms, R. W.: HIV-1 entry inhibitors: new targets, novel therapies, Immunol. Lett., (2003), 85:113-118; Davis, C. W., Doms, R. W.: HIV Transmission: Closing all the Doors, J. Exp. Med., (2004), 199:1037-1040; Hu, Q., Frank, I., Williams, V., Santos, J. J., Watts, P., Griffin, G. E., Moore, J. P., Pope, M., Shattock, R. J.: Blockade of attachment and fusion receptors inhibits HIV-1 infection of human cervical tissue, J. Exp. Med., (2004), 199:1065-1075]. Therefore, it was of interest to determine whether or not pomegranate juice inhibited the binding of the HIV-1 envelope glycoprotein gp120 to CD4, the common receptor for both the X4 and R5 viruses. Pretreatment of both gp120 IIIB and BaL by pomegranate juice inhibited subsequent binding of soluble labeled CD4 (FIG. 2). This suggested that one or more pomegranate juice ingredients bound strongly or irreversibly to the CD4 binding site on gp120. These results, obtained in an ELISA using gp120 immobilized on polystyrene plates, were confirmed in another assay in which both gp120 and CD4 were in soluble form (data not shown). In reverse experiments, pretreatment of CD4 with pomegranate juice failed to block subsequent gp120 binding. Other juices having anti-HIV-1 activity (blueberry, cranberry, grape and lime) failed to block gp120-CD4 binding.

To delineate sites on gp120 blocked by the pomegranate juice inhibitor(s), the inhibitory effect of pomegranate juice on binding to gp120 IIIB of antibodies to peptides derived from the amino acid sequence of gp120 was studied. The binding of antibodies to peptides (102-126), (303-338), (306-338), (361-392), (386-417), (391-425), (411-445) and (477-508) was significantly (≈50%) inhibited (FIG. 3). The binding to gp120 IIIB of monoclonal antibodies 9284 and 588D, specific for the gp120 V3 loop (residues 303-338) and the CD4 binding site, respectively [Skinner, M. A., Ting, R., Langlois, A. J., Weinhold, K. J., Lyerly, H. K., Javaherian, K., Matthews, T. J.: Characteristics of a neutralizing monoclonal antibody to the HIV envelope glycoprotein. AIDS Res. Hum. Retroviruses, (1988), 4:187-197; Laal, S., Zolla-Pazner, S.: Epitopes-of HIV-1 glycoproteins recognized by the human immune system, In Immunochemistry of AIDS, Chemical Immunology, Volume 56, Edited by Norrby E. Basel: Karger; 1993:91-111] was each inhibited by 97%. Some of the relevant peptides contain residues involved in CD4 binding [Kwong, P. D., Wyatt,. R., Robinson, J., Sweet, R. W., Sodroski, J., Hendrickson, W. A.: Structure of an HIV gp120 envelope glycoprotein in complex with the CD4 receptor and a neutralizing human antibody, Nature, (1998), 393:648-659; Xiang, S. H., Kwong, P. D., Gupta, R., Rizzuto, C. D., Casper, D. J., Wyatt, R., Wang, L., Hendrickson, W. A., Doyle, M. L., Sodroski, J.: Mutagenic stabilization and/or disruption of a CD4-bound state reveals distinct conformations of the human immunodeficiency virus type 1 gp120 envelope glycoprotein, J. Virol., (2002), 76:9888-9899; Pantophlet, R., Ollmann Saphire, E., Poignard, P., Parren, P. W. I., Wilson, I. A., Burton, D. R.: Fine mapping of the interaction of neutralizing and nonneutralizing monoclonal antibodies with the CD4 binding site of human immunodeficiency virus type 1 gp120, J. Virol., (2003), 77:642-658] while all discerned peptides include residues involved in coreceptor binding [Westervelt, P., Gendelman, H. E., Ratner, L.: Identification of a determinant within the human immunodeficiency virus 1 surface envelope glycoprotein critical for productive infection of primary monocytes, Proc. Natl. Acad. Sci. USA, (1991), 88:3097-3101; Westervelt, P., Trowbridge, D. B., Epstein, L. G., Blumberg, B. M., Li, Y., Hahn, B. H., Shaw, G. M., Price, R. W., Ratner, L.: Macrophage tropism determinants of human immunodeficiency virus type 1 in vivo, J. Virol., (1992), 66:2577-2582; Rizzuto, C. D., Wyatt, R., Hernandez-Ramos, N., Sun, Y., Kwong, P. D., Hendrickson, W. A., Sodroski, J.: A conserved HIV gp120 glycoprotein structure involved in chemokine receptor binding, Science, (1998), 80:1949-1953;,Cormier, E. G., Dragic, T.: The crown and stem of the V3 loop play distinct roles in human immunodeficiency virus type 1 envelope glycoprotein interactions with the CCR5 coreceptor, J. Virol., (2002), 76:8953-8957; Suphaphiphat, P., Thitithanyanont, A., Paca-Uccaralertkun, S., Essex, M., Lee, T-H: Effect of amino acid substitution of the V3 and bridging sheet residues in human immunodeficiency virus type 1 subtype C gp120 on CCR5 utilization, J. Virol., (2003), 77:3832-3837; Liu, S., Fan, S., Sun, Z.: Structural and functional characterization of the human CCR5 receptor in complex with HIV gp120 envelope glycoprotein and CD4 receptor by molecular modeling studies, J. Mol. Model (Online) (2003), 9:329-336]. The locations of the peptides and of residues involved in receptor/coreceptor binding on the X-ray crystallographic structure of gp120 are shown in FIG. 4. These results suggest that the pomegranate juice inhibitor(s) may also block gp120-coreceptor binding. Separation of Anti-HIV-1 Inhibitor(s)from Pomegranate Juice Pomegranate juice is intensely colored. Therefore, it cannot be directly formulated into a microbicide since it would stain clothing, which is unacceptable. Attempts were made to separate or isolate the active ingredient(s) from pomegranate juice. After striving intermittently for over four years to accomplish this, it was discovered that the inhibitor(s) of gp120-CD4 binding can be adsorbed effectively (≧99%) onto a selected brand of corn starch (FIG. 5), resulting in a nearly colorless product, designated as PJ-S21. PJ-S21, suspended in water or unbuffered 0.14 M NaCl had a pH of 3.2, compatible with the acidic vaginal environment in which it would remain stabile after application (see herein). Inhibitors of gp120-CD4 binding could be eluted from PJ-S21 by extraction with ethanol/acetone 5:4. Drying of the extract followed by gravimetry indicated that the extract contains 3.17 mg solids per gram of PJ-S21.

PJ-S21, to the same extent as the original pomegranate juice, inhibited the binding of gp120 IIIB-CD4 complexes to cells expressing CXCR4, as determined by flow cytometry (FIG. 6). Similarly, binding of a gp120 BaL-CD4 fusion protein to cells expressing CCR5 was blocked by pomegranate juice and PJ-S21, as determined by a cell based ELISA [Zhao, Q., Alespeiti, G., Debnath, A. K.: A novel assay to identify entry inhibitors that block binding of HIV-1 gp120 to CCR5, Virol., 326:299-309]; (FIG. 7). Thus, PJ-S21 is an inhibitor of both X4 and R5 virus binding to the cellular receptor CD4 and coreceptors CXCR4/CCR5. PJ-S21 also inhibited gp120 binding to PBMCs, as determined by flow cytometry (FIGS. 8A and 8B). To confirm that PJ-S21 functions as a virus entry inhibitor, the complex was added to cells at time intervals before and after infection of cells by HIV-1 IIIB and BaL, respectively. Results shown in FIG. 9 demonstrate that PJ-S21 interferes with early steps of the virus replicative cycle.

To be considered as a topical microbicide, PJ-S21 must be formulated to withstand storage in a tropical environment. Accelerated thermal stability studies revealed that a water suspension of PJ-S21 maintained only 4, 11, and 33%, respectively, of its original activity (measured by inhibition of gp120-CD4 binding) when stored for 30 minutes at 60° C., and one week at 50° C. or 40° C. On the other hand, a dried PJ-S21 powder remained fully active after storage at 50° C. for 12 weeks (the longest time used in the evaluation). Consequently, anhydrous formulations may be desirable.

Three such formulations were prepared: two kinds of suppositories, melting at 37° C., and a tablet (the compositions of which are described herein). The inhibitory activity of PJ-S21 was fully preserved after 12 weeks storage at 50° C. within tablets, and at 30° C. within the suppositories (the highest temperature considered to prevent melting). Data showing the inhibition of infection by HIV-1 IIIB and BaL respectively, by PJ-S21 and its formulations (except the tablets which also contain anti-HIV-1 inhibitors other than PJ-S21, i.e., Carbopol 974P [Neurath, A. R., Strick, N., Li, Y-Y: Anti-HIV-1 activity of anionic polymers: A comparative study of candidate microbicides, BMC Infect. Dis., (2002), 2:27]) are summarized in FIG. 10. Their inhibitory activities against HIV-1 IIIB and BaL were similar, unlike the inhibitory activities of the original pomegranate juices (FIG. 1). These formulations were also virucidal, albeit at concentrations higher than those sufficient for inhibition of infection. These experiments also revealed that PJ-S21 was not cytotoxic under the experimental conditions used. The inhibitory/virucidal activities were maintained in the presence of seminal fluid (SF) at a 1:1 (w/w) ratio of SF to PJ-S21 (data not shown).

A microbicide can be considered potentially successful only if it has antiviral activity against primary virus isolates belonging to distinct virus clades and phenotypes. PJ-S21 meets this requirement since it inhibited infection by primary HIV-1 strains of all clades tested having R5 and X4R5 (=dual-tropic) phenotypes (Table 1).

PJ-S21 can be classified as an “AAAA” candidate microbicide, namely acceptable, accessible, affordable and accelerative in transition from development to marketing. Thus, PJ-S21 would be expected to circumvent some problems associated with antiretroviral drugs and possibly some of the other candidate microbicides, i.e., uncertainty related to potential side effects, investment and time needed to establish inexpensive large scale production, and monopoly of supply.

TABLE 1
Inhibitory activity of PJ-S21 on infection by primary HIV-1 strains

| | Subtype, | ED₅₀ * | ED₉₀ * |
|---|---|---|---|
| Primary strain | Coreceptor use | ma/ml | ma/ml |
| 92RW008 | A. R5 | 0.50 ± 0.05 | 2.76 ± 0.28 |
| 94UG103 | A. X4R5 | 1.42 ± 0.54 | 3.42 ± 0.98 |
| 92US657 | B. R5 | 0.62 ± 0.11 | 2.86 ± 0.33 |
| 93IN101 | C. R5 | 3.56 ± 1.10 | 8.87 ± 2.55 |
| 93MW959 | C. R5 | 1.02 ± 0.19 | 3.54 ± 0.90 |
| 92UG001 | D. X4R5 | 0.62 ± 0.17 | 2.94 ± 0.85 |
| 93THA051 | E. X4R5 | 0.86 ± 0.01 | 4.09 ± 0.08 |
| 93BR020 | F. X4R5 | 4.25 ± 0.78 | 8.31 ± 1.04 |
| RU570 | G. R5 | 0.42 ± 0.09 | 1.54 ± 0.16 |
| BCF02 | Group O. R5 | 0.59 ± 0.29 | 3.92 ± 0.27 |

* ED₅₀₍₉₀₎ = effective dose(s) for 50% (90%) inhibition of infection

One gram of PJ-S21 contains approximately 3.2 mg of the inhibitors adsorbed to starch from pomegranate juice.

It will be appreciated that the instant specification is set forth by way of illustration and not limitation, and that various modifications and changes may be made without departing from the spirit and scope of the present invention.

What is claimed is:

1. A complex comprising a starch and an active anti-HIV-1 or anti-HIV-2 ingredient of pomegranate juice that is adsorbed on the starch when said starch is in a water insoluble form, said complex inhibits HIV-1 or HIV-2 infection, and said complex blocks the binding of HIV-1 or HIV-2 to the CD4 receptor and the CCR5 and CXCR4 coreceptors.

2. The complex according to claim 1, wherein the complex inhibits HIV-1 infection.

3. The complex according to claim 1, wherein the complex inhibits HIV-2 infection.

4. The complex according to claim 1, wherein the complex is produced by combining 100 to 250 mg of the starch with 1 ml of pomegranate juice.

5. A pharmaceutical composition comprising a pharmaceutically effective anti-HIV-1 or anti-HIV-2 amount of the complex according to claim 1 and a pharmaceutically acceptable carrier.

6. A method for preventing HIV-1 or HIV-2 infection in a human comprising administering to a mucous membrane of a human a pharmaceutically effective anti-HIV-1 or anti-HIV-2 amount of the complex according to claim 1.

7. The method according to claim 6, wherein the method is for preventing HIV-1 infection.

8. The method according to claim 6, wherein the method is for preventing HIV-2 infection.

9. The method according to claim 6, wherein the administering is carried out by a vaginal administration.

10. The method according to claim 6, wherein the administering is carried out by a rectal administration.

11. A method for preventing HIV-1 or HIV-2 infection in a human comprising administering to a mucous membrane of a human a pharmaceutically effective anti-HIV-1 or anti-HIV-2 amount of the pharmaceutical composition according to claim 5.

12. The method according to claim 11, wherein the method is for preventing HIV-1 infection.

13. The method according to claim 11, wherein the method is for preventing HIV-2 infection.

14. The method according to claim 11, wherein 0.5 to 3 g of the complex are administered.

15. The method according to claim 11, wherein the administering is carried out by a vaginal administration.

16. The method according to claim 11, wherein the administering is carried out by a rectal administration.
